(19) United States
(12) Patent Application Publication
(10) Pub. No.: US 2025/0236916 A1
(43) Pub. Date: Jul. 24, 2025

(54) ENRICHMENT OF ABERRANTLY MODIFIED DNA

(71) Applicant: GUARDANT HEALTH, INC., Palo Alto, CA (US)

(72) Inventor: Andrew KENNEDY, San Diego, CA (US)

(21) Appl. No.: 18/968,047

(22) Filed: Dec. 4, 2024

Related U.S. Application Data
(63) Continuation of application No. PCT/US2023/069373, filed on Jun. 29, 2023.
(60) Provisional application No. 63/367,462, filed on Jun. 30, 2022.

(51) Int. Cl. C12Q 1/6886; C12N 9/22; C12N 15/113; C12Q 1/6869

(52) U.S. Cl. CPC C12Q 1/6886; C12N 9/226; C12N 15/113; C12Q 1/6869; C12N 2310/20; C12Q 2600/154

(57) ABSTRACT

Provided herein are methods of analyzing DNA comprising degrading forms of DNA sequences that are prevalent in healthy subjects; and detecting sequences that are not degraded. Some such methods facilitate detection of aberrant forms of DNA.

### CROSS-REFERENCE TO RELATED APPLICATIONS

The present application is a Continuation of International Patent Application No. PCT/US2023/069373, filed Jun. 30, 2022, which claims the benefit of U.S. Provisional Application No. 63/367,462, filed on Jun. 30, 2022, which is incorporated herein by reference for all purposes.

### FIELD OF THE INVENTION

The present disclosure provides methods and compositions related to analysis of aberrantly modified DNA, such as aberrantly methylated cell-free DNA (cfDNA), in a sample using sequence-specific degradation. In some embodiments, the sample comprises cfDNA from a subject having or suspected of having cancer and/or the sample comprises DNA from cancer cells. In some embodiments, aberrantly modified cfDNA in a sample is enriched by degrading DNA sequences comprising the modification that are prevalent in cfDNA from a healthy subject; and the remaining DNA comprising the modification is detected.

### INTRODUCTION AND SUMMARY

Cancer is responsible for millions of deaths per year worldwide. Early detection of cancer may result in improved outcomes because early-stage cancer tends to be more susceptible to treatment.

Improperly controlled cell growth is a hallmark of cancer that generally results from an accumulation of genetic and epigenetic changes, such as copy number variations (CNVs), single nucleotide variations (SNVs), gene fusions, insertions and/or deletions (indels), epigenetic variations including modification of cytosine (e.g., 5-methylcytosine, 5-hydroxymethylcytosine, and other more oxidized forms) and association of DNA with chromatin proteins and transcription factors. Thus, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. Hypermethylation can be associated with an aberrant loss of transcriptional capacity of involved genes and occurs at least as frequently as point mutations and deletions as a cause of altered gene expression. Furthermore, without wishing to be bound by any particular theory, cells in or around a cancer or neoplasm may shed more DNA than cells of the same tissue type in a healthy subject. The DNA from such cells may differ epigenetically from shed DNA in a healthy subject. As such, the distribution of epigenetically modified (e.g., methylated) DNA in certain DNA samples, such as cell-free DNA (cfDNA), may change upon carcinogenesis. Thus, sufficiently sensitive epigenetic (e.g., DNA methylation) profiling can be used to detect aberrant methylation in DNA of a sample.

Biopsies represent a traditional approach for detecting or diagnosing cancer in which cells or tissue are extracted from a possible site of cancer and analyzed for relevant phenotypic and/or genotypic features. Biopsies have the drawback of being invasive.

Detection of cancer based on analysis of body fluids (“liquid biopsies”), such as blood, is an intriguing alternative based on the observation that DNA from cancer cells is released into body fluids. A liquid biopsy is noninvasive (sometimes requiring only a blood draw). However, it has been challenging to develop accurate and sensitive methods for analyzing liquid biopsy material that provides detailed information regarding nucleobase modifications given the low concentration and heterogeneity of cell-free DNA. The contribution of DNA from cells in or around a cancer or neoplasm to a sample may be relatively small relative to the contribution from other cells, and the DNA contributed from other cells may be uninformative as to cancer status. Isolating and processing the fractions of cell-free DNA useful for further analysis in liquid biopsy procedures is an important part of these methods. Accordingly, there is a continued need for improved methods and compositions for analyzing cell-free DNA, e.g., in liquid biopsies.

Methods according to this disclosure can comprise degradation of DNA sequences that are, e.g., modified in healthy subjects and/or uninformative as to cancer status. The degradation can replace more laborious steps in existing DNA profiling methods; therefore, methods herein can provide shorter, more efficient, more cost-effective, and/or more sensitive methods of detecting aberrantly modified DNA. Furthermore, prior knowledge or identification of the sequences that are aberrantly modified is not required to carry out methods herein.

In some embodiments, DNA methylation comprises addition of a methyl group to a cytosine residue at a CpG dinucleotide (cytosine-phosphate-guanine dinucleotide (i.e., a cytosine followed by a guanine in a 5′->3′ direction of the nucleic acid sequence). In some embodiments, DNA methylation comprises addition of a methyl group to an adenine residue, such as in N6-methyladenine. In some embodiments, DNA methylation is 5-methylation (modification of the 5th carbon of the 6-carbon ring of cytosine). In some embodiments, 5-methylation comprises addition of a methyl group to the 5C position of the cytosine residue to create 5-methylcytosine (m5c or 5-mC or 5mC). In some embodiments, methylation comprises a derivative of m5c. Derivatives of m5c include, but are not limited to, 5-hydroxymethylcytosine (5-hmC or 5hmC), 5-formylcytosine (5-fC), and 5-caryboxylcytosine (5-caC). In some embodiments, DNA methylation is 3C methylation (modification of the 3rd carbon of the 6-carbon ring of the cytosine residue). In some embodiments, 3C methylation comprises addition of a methyl group to the 3C position of the cytosine residue to generate 3-methylcytosine (3mC). Methylation can also occur at non-CpG sites, for example, methylation can occur at a CpA, CpT, or CpC site. DNA methylation can change the activity of the methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The present disclosure aims to meet the need for improved analysis of cfDNA and/or provide other benefits. Accordingly, the following exemplary embodiments are provided.

Embodiment 1 is a method of analyzing DNA in a sample, the method comprising:
a) partitioning the sample into a plurality of subsamples by contacting the DNA with an agent that recognizes a modification associated with the DNA, the plurality comprising a first subsample and a second subsample, wherein the first subsample comprises DNA associated with the modification in a greater proportion than the second subsample;
b) sequence-specifically degrading a plurality of DNA sequences in the first subsample that comprise the modification and are prevalent in cell-free DNA (cfDNA) from a healthy subject, comprising contacting the first subsample with a modification-independent sequence-specific nuclease, thereby producing a treated sample; and
c) detecting the presence or absence of one or more DNA sequences associated with the modification in the treated sample.

Embodiment 2 is the method of embodiment 1, wherein the partitioning occurs prior to the degrading.

Embodiment 3 is a method of analyzing cfDNA in a sample, the method comprising:
a) contacting the sample or a subsample thereof with a MSRE, thereby degrading DNA comprising an unmethylated recognition site of the MSRE;
b) sequence-specifically degrading a plurality of DNA sequences having methylated sequences that are prevalent in cfDNA from a healthy subject and a plurality of sequences lacking CpG motifs, comprising contacting the sample with a modification-independent sequence-specific nuclease, thereby producing a treated sample; and
c) detecting the presence of absence of one or more DNA sequences in the treated sample.

Embodiment 4 is the method of the immediately preceding embodiment, wherein the methylation comprises cytosine methylation.

Embodiment 5 is the method of embodiment 3 or embodiment 4, further comprising partitioning the sample into a plurality of subsamples by contacting the DNA with an agent that recognizes a modification associated with the DNA, the plurality comprising a first subsample and a second subsample, wherein the first subsample comprises DNA associated with the modification in a greater proportion than the second subsample.

Embodiment 6 is the method of any one of the preceding embodiments, wherein the partitioning comprises partitioning on the basis of methylation level of the DNA.

Embodiment 7 is the method of any one of the preceding embodiments, wherein the modification is methylation.

Embodiment 8 is the method of the immediately preceding embodiment, wherein the methylation comprises cytosine methylation.

Embodiment 9 is the method of any one of embodiments 1, 2, or 5-8, wherein the partitioning comprises partitioning on the basis of hydroxymethylation level of the DNA.

Embodiment 10 is the method of the immediately preceding embodiment, wherein hydroxymethyls are labeled prior to partitioning, optionally wherein the label comprises a biotin, glucosyl, or sulfonyl.

Embodiment 11 is the method of any one of embodiments 1-3 or 7-10, wherein the modification is hydroxymethylation.

Embodiment 12 is the method of any one of the preceding embodiments, wherein the agent that recognizes a modification associated with the DNA is a methyl, hydroxymethyl, or labeled hydroxymethyl binding reagent.

Embodiment 13 is the method of the immediately preceding embodiment, wherein the methyl, hydroxymethyl, or labeled hydroxymethyl binding reagent is an antibody.

Embodiment 14 is the method of embodiment 12 or 13, wherein the methyl binding reagent specifically recognizes 5-methylcytosine, 5-hydroxymethylcytosine, biotinylated 5-hydroxymethylcytosine, glucosylated 5-hydroxymethylcytosine, or sulfonylated 5-hydroxymethylcytosine.

Embodiment 15 is the method of any one of embodiments 12-14, wherein the methyl binding reagent is immobilized on a solid support.

Embodiment 16 is the method of any one of the preceding embodiments, wherein partitioning comprises immunoprecipitation of methylated, hydroxymethylated, or labeled hydroxymethylated DNA.

Embodiment 17 is the method of any one of embodiments 1, 2, or 5-16, wherein the partitioning comprises partitioning on the basis of binding to a protein, optionally wherein the protein is a methylated protein, an acetylated protein, an unmethylated protein, an unacetylated protein; and/or optionally wherein the protein is a histone.

Embodiment 18 is the method of the immediately preceding embodiment, wherein the partitioning comprises contacting the collected cfDNA with a binding reagent which is specific for the protein and is immobilized on a solid support.

Embodiment 19 is the method of any one of the preceding embodiments, wherein the degrading comprises contacting the second subsample with a modification-independent sequence-specific nuclease, thereby producing a second treated sample.

Embodiment 20 is the method of any one of embodiments 1, 2, or 5-19, comprising contacting one or more of the plurality of subsamples with a methylation-sensitive restriction enzyme (MSRE), thereby degrading DNA comprising an unmethylated recognition site of the MSRE.

Embodiment 21 is the method of the immediately preceding embodiment, wherein the contacting one or more of the plurality of subsamples with the MSRE occurs after the partitioning.

Embodiment 22 is the method of embodiment 20 or 21, wherein the contacting one or more of the plurality of subsamples with the MSRE occurs prior to the degrading.

Embodiment 23 is the method of embodiment 20 or 21, wherein the contacting one or more of the plurality of subsamples with the MSRE occurs after the degrading.

Embodiment 24 is the method of embodiment 20 or 21, wherein the contacting one or more of the plurality of subsamples with the MSRE occurs simultaneously with the degrading.

Embodiment 25 is the method of any one of embodiments 21-24, wherein the first subsample is contacted with the MSRE.

Embodiment 26 is the method of any one of embodiments 20-25, wherein the contacting a sample or subsample with the MSRE occurs prior to the sequence-specific degrading.

Embodiment 27 is the method of any one of the preceding embodiments, wherein the modification-independent sequence-specific nuclease is a CRISPR nuclease.

Embodiment 28 is the method of the immediately preceding embodiment, wherein the CRISPR nuclease is a Cas12a, Cas12b, or a CasX nuclease.

Embodiment 29 is the method of embodiment 27, wherein the CRISPR nuclease is a Cas9 nuclease.

Embodiment 30 is the method of the immediately preceding embodiment, wherein the Cas9 nuclease is a multi-turnover Cas9 nuclease.

Embodiment 31 is the method of embodiment 29, wherein the Cas9 nuclease is a Streptococcus pyogenes Cas9 nuclease or a variant thereof.

Embodiment 32 is the method of embodiment 29, wherein the Cas9 nuclease is a Staphylococcus aureus Cas9 nuclease or a variant thereof.

Embodiment 33 is the method of any one of embodiments 29-32, wherein the Cas9 nuclease is a high-fidelity variant.

Embodiment 34 is the method of any one of the preceding embodiments, wherein the sequence-specifically degrading comprises contacting the DNA with a plurality of guide RNAs.

Embodiment 35 is the method of the immediately preceding embodiment, wherein at least one guide RNA comprises one or more modifications.

Embodiment 36 is the method of the immediately preceding embodiment, wherein the one or more modifications comprise a phosphorothioate internucleoside linkage, a 2′-substitution, or a UNA, LNA, cEt, or ENA nucleotide sugar.

Embodiment 37 is the method of the immediately preceding embodiment, wherein the 2′ substitution is a 2′-fluoro, 2′-hydro, 2′-O-methoxyethyl, or 2′-O-alkyl.

Embodiment 38 is the method of any one of embodiments 34-37, wherein at least one guide RNA is an sgRNA.

Embodiment 39 is the method of any one of embodiments 34-38, wherein at least one guide RNA specifically binds to DNA comprising a CpG motif that is methylated in cfDNA from healthy tissue or from a healthy subject.

Embodiment 40 is the method of any one of embodiments 34-39, wherein at least one guide RNA specifically binds to a DNA sequence lacking a CpG dinucleotide.

Embodiment 41 is the method of any one of embodiments 34-39, wherein each guide RNA of the plurality of guide RNAs is configured to specifically bind to a DNA sequence that comprises the modification and is prevalent in cell-free DNA (cfDNA) from a healthy subject.

Embodiment 42 is the method of any one of embodiments 34-40, wherein each guide RNA of the plurality of guide RNAs is configured to specifically bind to a DNA sequence that comprises the modification and is prevalent in cell-free DNA (cfDNA) from a healthy subject or to a DNA sequence lacking a CpG dinucleotide.

Embodiment 43 is the method of any one of embodiments 1-26, 34-37, or 39-42, wherein the modification-independent sequence-specific nuclease is an Argonaute nuclease.

Embodiment 44 is the method of any one of embodiments 1-26, 34-37, or 39-42, wherein the modification-independent sequence-specific nuclease is a zinc finger nuclease.

Embodiment 45 is the method of any one of embodiments 1-26, 34-37, or 39-42, wherein the modification-independent sequence-specific nuclease is a TALEN.

Embodiment 46 is the method of any one of the preceding embodiments, wherein the detecting step comprises sequencing.

Embodiment 47 is the method of the immediately preceding embodiment, wherein the detecting step comprises sequencing a plurality of target regions in at least one target region set.

Embodiment 48 is the method of any one of the preceding embodiments, further comprising enriching for one or more of the plurality of target regions in at least one target region set.

Embodiment 49 is the method of the immediately preceding embodiment, wherein the enriching comprises contacting the DNA with target-specific probes specific for the one or more of the plurality of target regions in at least one target region set.

Embodiment 50 is the method of any one of embodiments 47-49, wherein the at least one target region set comprises target regions that are not prevalent in methylated form in cfDNA from a healthy subject or not prevalent in methylated form in healthy tissue.

Embodiment 51 is the method of embodiment 47-50, wherein the at least one target region set comprises target regions that are prevalent in methylated form in tissue that does not substantially contribute to cfDNA in a healthy subject.

Embodiment 52 is the method of any one of embodiments 47-51, wherein the at least one target region set comprises target regions that are prevalent in methylated form in a cancerous tissue.

Embodiment 53 is the method of any one of embodiments 47-52, wherein the at least one target region set comprises a sequence-variable target region set and an epigenetic target region set.

Embodiment 54 is the method of any one of embodiments 47-53, wherein the at least one target region set comprises a hypermethylation variable target region set.

Embodiment 55 is the method of the immediately preceding embodiment, wherein the hypermethylation variable target region set comprises regions having a higher degree of methylation in at least one type of tissue than the degree of methylation in cfDNA from a healthy subject.

Embodiment 56 is the method of any one of embodiments 47-55, wherein the at least one target region set comprises a hypomethylation variable target region set.

Embodiment 57 is the method of the immediately preceding embodiment, wherein the hypomethylation variable target region set comprises regions having a lower degree of methylation in at least one type of tissue than the degree of methylation in cfDNA from a healthy subject.

Embodiment 58 is the method of any one of embodiments 47-57, wherein the at least one target region set comprises a methylation control target region set.

Embodiment 59 is the method of any one of embodiments 47-57, wherein the at least one target region set comprise a fragmentation variable target region set.

Embodiment 60 is the method of the immediately preceding embodiment, wherein the fragmentation variable target region set comprises transcription start site regions.

Embodiment 61 is the method of embodiment 59 or 60, wherein the fragmentation variable target region set comprises CTCF binding regions.

Embodiment 62 is the method of any one of embodiments 53-61, wherein the sequence-variable target region set comprises at least one sequence that is not prevalent in cfDNA from a healthy subject.

Embodiment 63 is the method of any one of embodiments 46-62, wherein the sequencing comprises sequencing genes or portions thereof of genes selected from Table 1, Table 2, Table 3, Table 4, and/or Table 5.

Embodiment 64 is the method of any one of embodiments 46-63, wherein the sequencing comprises sequencing all of the DNA sequences in the treated sample.

Embodiment 65 is the method of any one of the preceding embodiments, wherein 20-250,000 sequences are degraded.

Embodiment 66 is the method of any one of the preceding embodiments, wherein 50-100,000 sequences are degraded.

Embodiment 67 is the method of the immediately preceding embodiment, wherein 100-10,000 sequences are degraded.

Embodiment 68 is the method of any one of the preceding embodiments, wherein the sequences that are degraded comprise repetitive elements, optionally wherein the repetitive elements comprise SINEs, LINEs, and/or Alu elements.

Embodiment 69 is the method of any one of embodiments 1-45, wherein the detecting step comprises performing qPCR.

Embodiment 70 is the method of any one of the preceding embodiments, wherein the DNA is collected from a test subject.

Embodiment 71 is the method of any one of the preceding embodiments, wherein the DNA comprises cfDNA obtained from a test subject.

Embodiment 72 is the method of embodiments 70 or 71, wherein the DNA comprises DNA obtained from a tissue sample of the test subject.

Embodiment 73 is the method of the immediately preceding embodiment, wherein the tissue sample is a biopsy, a fine needle aspirate, or a formalin-fixed paraffin-embedded tissue sample.

Embodiment 74 is the method of any one of the preceding embodiments, further comprising ligating barcode-containing adapters to the DNA, optionally wherein the ligating occurs before or simultaneously with amplification of the DNA.

Embodiment 75 is the method of the immediately preceding embodiment, wherein the plurality of sequences that are sequence-specifically degraded comprises sequences comprising barcode-containing adapter dimer junctions.

Embodiment 76 is the method of the immediately preceding embodiment, comprising contacting the DNA with a plurality of guide RNAs configured to specifically bind to each possible adapter dimer junction.

Embodiment 77 is the method of any one of the preceding embodiments, wherein the DNA is amplified before the detecting.

Embodiment 78 is the method of any one of the preceding embodiments, wherein the sequence specific degrading occurs after the ligating barcode-containing adapters to the DNA and
(a) prior to the partitioning the sample into a plurality of subsamples by contacting the DNA with an agent that recognizes a modification associated with the DNA;
(b) after the partitioning the sample into a plurality of subsamples by contacting the DNA with an agent that recognizes a modification associated with the DNA;
(c) prior to the contacting the sample or a subsample thereof with a MSRE;
(d) after the contacting the sample or a subsample thereof with a MSRE;
(c) prior to the step of amplifying the DNA before the detecting;
(f) after the step of amplifying the DNA before the detecting; or
(g) any combination of (a) with any one or two of (c)-(f); (b) with any one or two of (c)-(f); (c) with any one or two of (a), (b), (c), and (f); (d) with any one or two of (a), (b), (e), and (f); (e) with any one or two of (a)-(d); or (f) with any one or two of (a)-(d).

Embodiment 79 is the method of any one of the preceding embodiments, comprising partitioning the sample, wherein DNA molecules from the first subsample and DNA molecules from the second subsample are differentially tagged.

Embodiment 80 is the method of the immediately preceding embodiment, wherein DNA molecules from the first subsample and DNA molecules from the second subsample are sequenced in the same sequencing cell.

Embodiment 81 is the method of embodiment 79 or 80, comprising differentially tagging and pooling the first subsample and second subsample.

Embodiment 82 is the method of any one of embodiments 79-81, wherein the DNA of the first subsample and the DNA of the second subsample are differentially tagged; and after differential tagging, a portion of DNA from the second subsample is added to the first subsample or at least a portion thereof, thereby forming a pool.

Embodiment 83 is the method of the immediately preceding embodiment, wherein the pool comprises less than or equal to about 45%, 40%, 35%, 30%, 25%, 20%, 15%, 10%, or 5% of the DNA of the second subsample.

Embodiment 84 is the method of the immediately preceding embodiment, wherein the pool comprises about 70-90%, about 75-85%, or about 80% of the DNA of the second subsample.

Embodiment 85 is the method of any one of embodiments 82-84, wherein the pool comprises substantially all of the DNA of the first subsample.

Embodiment 86 is the method of any one of the preceding embodiments, comprising partitioning the sample into a plurality of subsamples, wherein the plurality of subsamples comprises a third subsample, which comprises DNA with a cytosine modification in a greater proportion than the second subsample but in a lesser proportion than the first subsample.

Embodiment 87 is the method of the immediately preceding embodiment, wherein the method further comprises differentially tagging the third subsample.

Embodiment 88 is the method of any one of the preceding embodiments, wherein the first subsample is enriched for one or more target region sets before sequence-specifically degrading the plurality of DNA sequences in the first subsample that comprise the modification and are prevalent in cell-free DNA.

Embodiment 89 is the method of any one of the preceding embodiments, wherein a plurality of first subsamples are pooled before sequence-specifically degrading the plurality of DNA sequences that comprise the modification and are prevalent in cell-free DNA, optionally wherein the plurality of first subsamples are from different subjects and/or are distinguishably tagged with sample tags.

Embodiment 90 is the method of any one of the preceding embodiments, further comprising determining a likelihood that the subject has cancer.

Embodiment 91 is the method of any one of embodiments 45-90, wherein the sequencing generates a plurality of sequencing reads; and the method further comprises mapping the plurality of sequence reads to one or more reference sequences to generate mapped sequence reads, and processing the mapped sequence reads corresponding to the sequence-variable target region set and to the epigenetic target region set to determine the likelihood that the subject has cancer.

Embodiment 92 is the method of any one of embodiments 70-91, wherein the test subject was previously diagnosed with a cancer and received one or more previous cancer treatments.

Embodiment 93 is the method of the immediately preceding embodiment, wherein the cfDNA is obtained at one or more preselected time points following the one or more previous cancer treatments, and the detecting comprises sequencing the DNA sequences, whereby a set of sequence information is produced.

Embodiment 94 is the method of the immediately preceding embodiment, further comprising detecting a presence or absence of DNA originating or derived from a tumor cell at a preselected timepoint using the set of sequence information.

Embodiment 95 is the method of the immediately preceding embodiment, further comprising determining a cancer recurrence score that is indicative of the presence or absence of the DNA originating or derived from the tumor cell for the test subject, optionally further comprising determining a cancer recurrence status based on the cancer recurrence score, wherein the cancer recurrence status of the test subject is determined to be at risk for cancer recurrence when a cancer recurrence score is determined to be at or above a predetermined threshold or the cancer recurrence status of the test subject is determined to be at lower risk for cancer recurrence when the cancer recurrence score is below the predetermined threshold.

Embodiment 96 is the method of the immediately preceding embodiment, further comprising comparing the cancer recurrence score of the test subject with a predetermined cancer recurrence threshold, wherein the test subject is classified as a candidate for a subsequent cancer treatment when the cancer recurrence score is above the cancer recurrence threshold or not a candidate for a subsequent cancer treatment when the cancer recurrence score is below the cancer recurrence threshold.

### BRIEF DESCRIPTION OF THE DRAWINGS

FIG. 1 illustrates an exemplary workflow according to certain embodiments of the disclosure. In FIG. 1, a black circle represents a 5mC at a CpG site that is not commonly methylated in cfDNA obtained from healthy subjects, and is not targeted for degradation by a sequence-specific nuclease. A black star represents a 5mC at a CpG site that is commonly methylated in cfDNA obtained from healthy subjects, and is targeted for degradation by a sequence-specific nuclease. The workflow begins with a blood sample, in which cfDNA is isolated from the blood sample; the cfDNA is partitioned based on methylation level, then the sample is subjected to molecular barcoding (“adapter/barcode ligation”); adapter/barcode-ligated sequences of the high methylation partition that are prevalent in samples from healthy subjects are subjected to CRISPR mediated degradation of target sequences (resulting in a reduction in the number of adapter/barcode-ligated molecules containing a CpG site that is commonly methylated in cfDNA obtained from healthy subjects, such that fewer molecules marked with black stars remain after this step); and DNA molecules of the sample are (in any suitable order) amplified and sequenced. The step of CRISPR mediated degradation of target sequences can occur at various times after adapter/barcode ligation. For example, in some embodiments, the step of CRISPR mediated degradation of target sequences occurs after a step of contacting the DNA with a methylation-sensitive nuclease and prior to sequencing. In some embodiments, the step of CRISPR mediated degradation of target sequences occurs after the amplification step and prior to sequencing.

FIG. 2 is a schematic diagram of an example of a system suitable for use with some embodiments of the disclosure.

FIG. 3 illustrates an exemplary workflow according to certain embodiments of the disclosure beginning with cfDNA isolated from a blood sample. The cfDNA is partitioned into at least two partitions based on methylation level, a hypermethylated partition and a hypomethylated partition, then adapters are added during library preparation. Unmethylated DNA present in the hypermethylated partition is digested using a MSRE, then DNA that is methylated in healthy subjects is sequence-specifically degraded using a CRISPR system, followed by amplification of the remaining DNA. The DNA present in the hypomethylated partition is processed, including adapter addition and amplification, then enriched for target regions using a target region probe set. The subsamples of the partitions are combined before sequencing and analysis.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, “CpG motif” is a contiguous DNA sequence comprising at least one CpG dinucleotide. In some embodiments, a CpG motif comprises one CpG dinucleotide. In some embodiments, a CpG motif comprises two CpG dinucleotides. In some embodiments, a CpG motif comprises a sequence that is specifically recognized by a sequence-specific nuclease. In some embodiments, a CpG motif comprises the recognition sequence of a methylation-sensitive nuclease or a methylation-dependent nuclease. The terms “CpG dinucleotide” and “CpG site” are used interchangeably herein.

As used herein, “prevalent” in the context of DNA sequences in a sample (e.g., of cfDNA) means present at a detectable level. In some embodiments, prevalent sequences in a sample are detectable in a majority of or all samples of a certain sample type. For example, methylated sequences prevalent in cfDNA from a healthy subject are methylated sequences that are detectable by standard techniques in the art in a sample from a healthy subject. In some such embodiments, methylated sequences prevalent in cfDNA from a healthy subject are thus not associated with a disease or a likelihood or risk of developing the disease.

As used herein, a modification is “associated with” DNA when the modification is a covalent modification of the DNA or the modification is a covalent modification of a protein (e.g., histone) bound to the DNA.

As used herein, “partitioning” of nucleic acids, such as DNA molecules, means separating, fractionating, or sorting a sample or population of nucleic acids into a plurality of subsamples or subpopulations of nucleic acids based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

A “converted nucleobase” is a nucleobase having an altered base pairing specificity, wherein the original base pairing specificity of the nucleobase was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. As used herein, a “converted sample” is a sample comprising DNA comprising at least one converted nucleobase.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage. Thus, a combination, combinations, or combination thereof refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Specifically binds” in the context of an oligonucleotide, such as a guide RNA, and a nucleic acid comprising a sequence that is partially or completely complementary to the oligonucleotide means that under appropriate hybridization conditions, the oligonucleotide hybridizes to the complementary sequence to form a stable oligonucleotide: complementary sequence hybrid, while at the same time formation of stable oligonucleotide: non-complementary sequence hybrids is minimized. Thus, an oligonucleotide hybridizes to a complementary sequence to a sufficiently greater extent than to a non-complementary sequence. Appropriate hybridization conditions are well-known in the art, may be predicted based on sequence composition, or can be determined by using routine testing methods (see, e.g., Sambrook et al., Molecular Cloning, A Laboratory Manual· 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

A “target region set” refers to a plurality of genomic loci comprising regions that share at least one common feature. In some embodiments, a target region set is identified by the at least one common feature. For example, a hypermethylation variable target region set comprises regions of DNA that are hypermethylated.

“Sequence-variable target region set” refers to a target region set comprising target regions that may exhibit changes in sequence, such as nucleotide substitutions (i.e., single nucleotide variations), insertions, deletions, or gene fusions or transpositions, in abnormal cells, such as neoplastic cells (e.g., tumor cells and cancer cells), relative to normal cells.

“Epigenetic target region set” refers to a target region set comprising target regions that may show sequence-independent changes in abnormal cells, such as neoplastic cells (e.g., tumor cells and cancer cells), relative to normal cells or that may show sequence-independent changes in cfDNA from subjects having cancer relative to cfDNA from healthy subjects. Examples of sequence-independent changes include, but are not limited to, changes in methylation (increases or decreases), nucleosome distribution, cfDNA fragmentation patterns, CCCTC-binding factor (“CTCF”) binding, transcription start sites, and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions.

A nucleic acid is “produced by a tumor” or is “circulating tumor DNA” (“ctDNA”) if it originated from a tumor cell. Tumor cells are neoplastic cells that originated from a tumor, regardless of whether they remain in the tumor or become separated from the tumor (as in the cases, e.g., of metastatic cancer cells and circulating tumor cells).

The term “methylation” in the context of a nucleic acid molecule refers to addition of a methyl group to a nucleobase in the nucleic acid molecule. In some embodiments, methylation refers to addition of a methyl group to a cytosine at a CpG site (cytosine-phosphate-guanine site (i.e., a cytosine followed by a guanine in a 5′->3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The term “epigenetic status” refers to a certain level or extent of a sequence-independent variable that may be present in a DNA sequence. In some embodiments, the epigenetic status of a DNA sequence refers to the extent or level of methylation, nucleosome distribution, cfDNA fragmentation pattern, CCCTC-binding factor (“CTCF”) binding, transcription start site, or regulatory protein binding region of the sequence. Epigenetic statuses thus include, but are not limited to, hypermethylation, hypomethylation, and the presence of absence of CTCF binding sites or transcription start sites. The epigenetic status of a sequence may be a “reference epigenetic status” that can be used for comparison to the epigenetic status of the corresponding sequence in other DNA molecules. An example of a reference epigenetic status is a status that is prevalent in samples obtained from healthy subjects and is not associated with cancer.

As used herein, “methylation status” refers to the presence or absence of a methyl group on a DNA nucleobase (e.g. cytosine) at a particular genomic position in a nucleic acid, the degree of methylation of a nucleic acid (e.g., high, low, intermediate, or unmethylated), or the number of nucleotides methylated in a particular nucleic acid molecule. A nucleic acid “in methylated form” means that it comprises a sequence containing a methylated DNA nucleobase, e.g., a methylated cytosine in a CpG dinucleotide.

As used herein, “consensus sequences” refer to sequences derived from redundant sequences of a parent molecule intended to represent the sequence of the original parent molecule. Consensus sequences include the base identity at a single position. In some embodiments, consensus sequence can represent a single nucleotide base at a particular genomic position. In some embodiments, consensus sequence can represent a string of nucleotide bases at a plurality of genomic positions. Consensus sequences can be produced by voting (wherein each majority nucleotide, e.g., the most commonly observed nucleotide at a given base position, among the sequences is the consensus nucleotide) or other approaches such as comparing to a reference genome. Consensus sequences can be produced by, e.g., tagging original parent molecules with unique or non-unique molecular tags, which allow tracking of the progeny sequences (e.g., after amplification) by tracking of the tag and/or use of sequence read internal information. Examples of tagging or barcoding, and uses of tags or barcodes, are provided herein and, for example, U.S. Patent Pub. Nos. 2015/0368708, 2015/0299812, 2016/0040229, and 2016/0046986, each of which is entirely incorporated herein by reference.

As used herein, “sequence-specific nuclease” means a nuclease that cleaves only nucleic acid sequences that comprise a particular sequence or consensus sequence. In some embodiments, sequence-specific nucleases cleave only nucleic acid sequences that comprise a particular sequence or consensus sequence that is at least 15, at least 16, or at least 17 nucleotides in length. In some embodiments, a sequence-specific nuclease is “modification-independent” and cleaves a particular sequence independent of the presence of modifications, such as methylation. In some embodiments, a sequence-specific nuclease is “modification-dependent” and cleaves sequences that comprise a particular sequence or consensus sequence and further comprise (or do not comprise) one or more nucleobase modifications, such as methylation. In some embodiments, modification independent sequence-specific nucleases bind to a guide RNA that hybridizes to or near to the sequence to be cleaved by the nuclease. Examples of sequence-specific nucleases include, but are not limited to, CRISPR (e.g., a Cas nuclease such as Cas9), Argonaute, TALEN, and zinc finger nucleases. A “variant” sequence-specific nuclease comprises at least one modification to at least one amino acid compared to the sequence-specific nuclease from which it was derived, has at least 80% sequence identity (e.g., at least 85%, 90%, 95%, 98%, or 99% sequence identity) to the sequence-specific nuclease from which it was derived, and retains at least some or enhanced nuclease function. The modification may be a natural or non-natural an amino acid substitution, deletion, or insertion.

As used herein, “sequence-specific degradation” or “sequence-specifically degrading” means degradation that depends on the sequence of the degraded site and is independent of modifications. Degrading includes any form of nucleolysis, such as endonucleolysis or cleavage of a nucleic acid.

As used herein, “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

As used herein, “methylation sensitive restriction enzyme” or “MSRE” refers to a methylation sensitive nuclease that is a restriction enzyme. An MSRE is sensitive to the methylation status of the DNA (e.g. cytosine methylation), i.e., the presence or absence of methyl group in a nucleotide base in its recognition sequence alters the rate at which the enzyme cleaves the DNA. In some embodiments, the methylation sensitive restriction enzymes do not cleave the DNA if a particular nucleotide base is methylated at the recognition sequence. For example, HpaII is a methylation sensitive restriction enzyme with a recognition sequence “CCGG” and it does not cleave DNA if the second cytosine in the recognition sequence is methylated.

As used herein, “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

As used herein, “methylation-dependent restriction enzyme” or “MDRE” refers to a methylation dependent nuclease that is a restriction enzyme. An MDRE is dependent on methylation of the DNA (e.g. cytosine methylation) i.e., the presence or absence of methyl group in a nucleotide base alters the rate at which the enzyme cleaves the DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR (N9)” and it does not cleave DNA if the absence of the methylated cytosine (mC) in the recognition sequence.

The terms “agent that recognizes a modified nucleobase in DNA” refers to a molecule or reagent that binds to or detects one or more modified nucleobases in DNA. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded.

“Or” is used in the inclusive sense, i.e., equivalent to “and/or,” unless the context requires otherwise.

### II. Exemplary Methods

### A. Sequence-Specifically Degrading a Plurality of Nucleic Acid Sequences

Methods disclosed herein comprise a step of sequence-specifically degrading a plurality of nucleic acid sequences comprising a modification. In some embodiments, the sequence-specific degrading comprises contacting the nucleic acids in a sample or subsample thereof with a sequence-specific nuclease. The modified sequences that are degraded are prevalent in nucleic acid sequences obtained from healthy subjects. In some embodiments, the nucleic acid sequences are cfDNA sequences. In some embodiments, the modification is methylation. In some embodiments, the methylation comprises or consists of cytosine methylation. In some embodiments, methylated sequences that are degraded comprise a CpG site that is commonly methylated in cfDNA obtained from healthy subjects. In some embodiments, sequences that comprise a CpG site that is not commonly methylated is not degraded by a sequence-specific nuclease. In some embodiments, methods herein comprise additional elements or steps to deplete unmodified sequences and sequences comprising modifications other than methylation or the modification of interest that are prevalent in cfDNA obtained from healthy subjects, such as additional elements or steps disclosed herein. The combination of depleting sequences that are modified in cfDNA from healthy subjects and depleting sequences that are not modified or contain other modifications in cfDNA from healthy subjects can provide a treated sample enriched for DNA molecules that are prevalent only in subjects that are not healthy. In some embodiments, sequences remaining uncleaved after the sequence-specific degrading step are detected. Thus, some methods herein provide detection of the presence or absence of aberrantly modified nucleic acid sequences by specifically degrading normally modified nucleic acid sequences prevalent in samples obtained from healthy subjects. In some embodiments, the treated sample is enriched for modified sequences that are associated with cancer. In general, the methods described herein can facilitate more efficient usage of capacity in downstream analytical steps (e.g., detection of sequences of interest, such as by sequencing). This is achieved by degrading uninformative sequences, e.g., which bear a modification such as methylation in all, essentially all, or a majority of samples, so that such uninformative sequences do not participate in and/or consume capacity during the downstream analytical steps. In some embodiments, the uninformative sequences comprise repetitive DNA elements (e.g., SINEs, LINEs, and/or Alu elements) and/or DNA that is methylated in healthy cells in the erythroid lineage.

In some embodiments, the sequence-specific degrading comprises cleavage of both strands of double-stranded DNA, resulting in formation of a double-strand end. In some embodiments, the double-strand end is a blunt end. In some embodiments, the double-strand end comprises an overhang of one or more nucleosides.

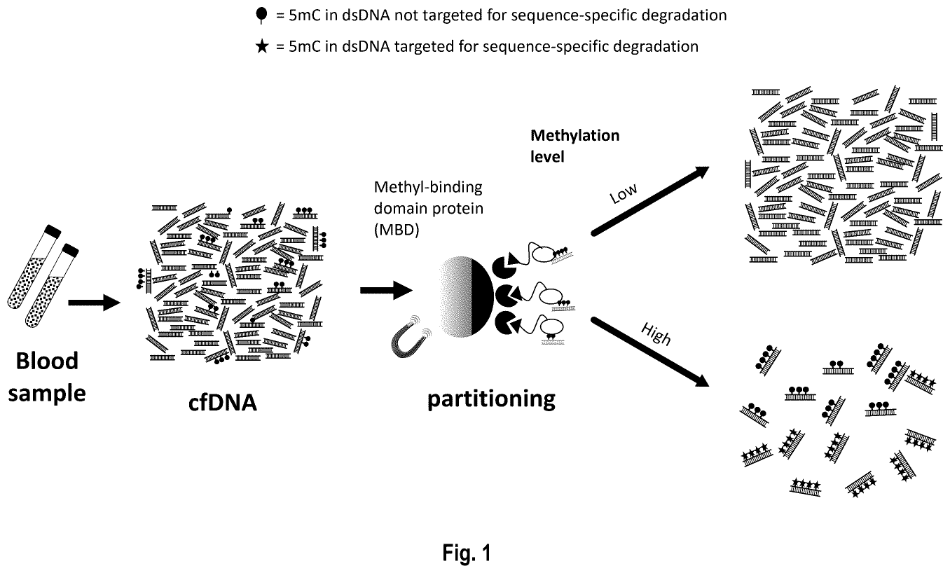

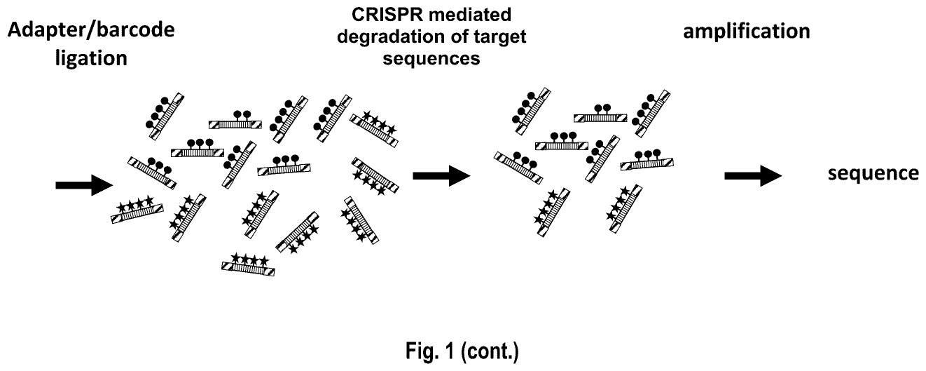

In some embodiments, the sequence-specific degrading is performed after partitioning the DNA based on methylation status. See, e.g., FIG. 1. In some such embodiments, the methods comprise contacting the DNA with a methyl binding domain (MBD) specific for methyl cytosine. In some embodiments, the sequence-specific degrading is performed on DNA of a hypermethylated partition but not performed on DNA of a hypomethylated partition. In some embodiments, the sequence specific degrading occurs before or after the adapters containing barcodes are ligated to DNA. In a particular embodiment, the sequence-specific degrading occurs after the adapters containing barcodes are ligated to DNA. In some embodiments, the sequence specific degrading occurs after the ligating barcode-containing adapters to the DNA and (a) prior to the partitioning the sample into a plurality of subsamples by contacting the DNA with an agent that recognizes a modification associated with the DNA; (b) after the partitioning the sample into a plurality of subsamples by contacting the DNA with an agent that recognizes a modification associated with the DNA; (c) prior to the contacting the sample or a subsample thereof with a MSRE; (d) after the contacting the sample or a subsample thereof with a MSRE; (c) prior to a step of amplifying the DNA before the detecting; (f) after a step of amplifying the DNA before the detecting; (prior to a step of enriching for one or more of the plurality of target regions in at least one target region set; after a step of enriching for one or more of the plurality of target regions in at least one target region set; (i) prior to two or more samples (or two or more subsamples) are pooled prior to sequencing the samples (such as in the same flow cell in a next-generation sequencing reaction), (j) after the two or more samples (or two or more subsamples) are pooled prior to sequencing the samples (such as in the same flow cell in a next-generation sequencing reaction), or (k) any combination of (a) with any one, two, three, or four of (c)-(j); (b) with any one, two, three, or four of (c)-(j); (c) with any one, two, three, or four of (a), (b), and (c)-(j); (d) with any one, two, three, or four of (a), (b), and (c)-(j); (c) with any one, two, three, or four of (a)-(d) and (g)-(j); (f) with any one, two, three, or four of (a)-(d) and (g)-(j); (g) with any one, two, three, or four of (a)-(f) and (i) or (j); (h) with any one, two, three, or four of (a)-(f) and (i)-(j); (i) with any one, two, three, or four of (a)-(h); or (j) with any one, two, three, or four of (a)-(h). In some embodiments, the DNA is then amplified, sequenced, and analyzed.

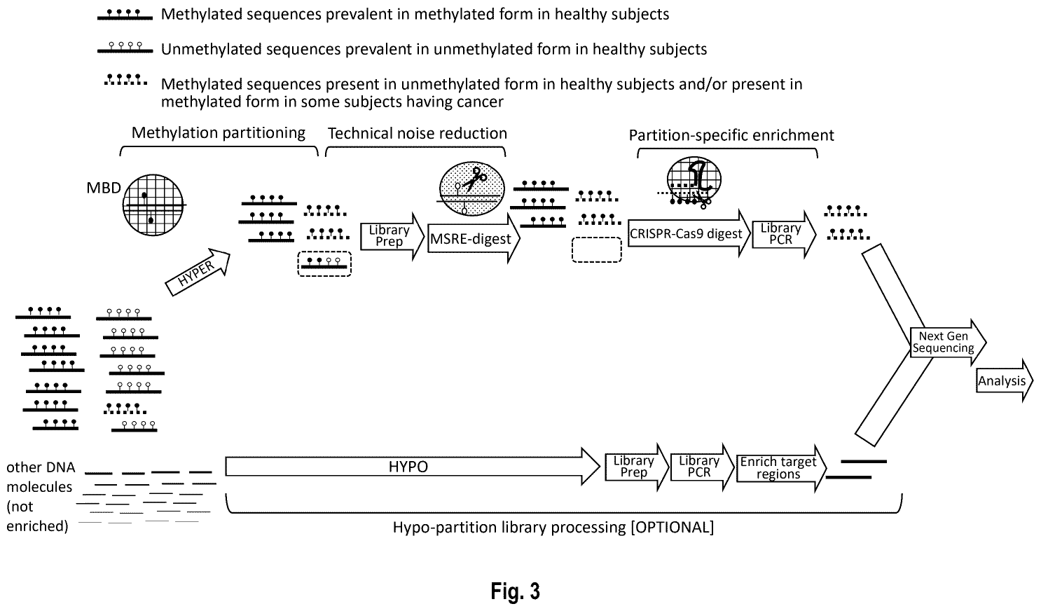

In some embodiments, the sequence-specific degrading is performed after partitioning the DNA based on methylated status, ligating adapters containing barcodes to the DNA, and degrading DNA of a hypothylated partition with a MSRE. Sec, e.g., FIG. 3. In some such embodiments, the DNA remaining after the sequence-specific degrading is amplified by PCR. In some embodiments, adapters containing barcodes are ligated to DNA of a hypomethylated partition, the DNA is then amplified by PCR and enriched using DNA or RNA probes of target region sets. In some such embodiments, DNA of the hypomethylated partition is not sequence-specifically degraded. In some embodiments, enriched DNA of the hypermethylated partition is combined with DNA remaining in the hypermethylated partition before sequencing and analysis.

### 1. Sequence-Specific Nucleases

In some embodiments, the sequence-specific degrading is performed by contacting nucleic acids, such as DNA, with a sequence-specific nuclease. In some embodiments, the sequence-specific nuclease is a modification-independent sequence-specific nuclease. Examples of modification-independent sequence-specific nucleases include but are not limited to CRISPR nucleases, TALENS, zinc fingers, and Argonaute nucleases.

In some embodiments, the modification-independent sequence-specific nuclease is a CRISPR nuclease. Exemplary CRISPR nucleases include Type II and Type V Cas nucleases, including Cas9, such as a Streptococcus pyogenes Cas9 nuclease or a variant thereof, a Staphylococcus aureus Cas9, or a variant thereof; Cas12, such as a Cas12a or Cas12b nuclease, or a variant thereof; and CasX nucleases or variants thereof. In some embodiments, a Cas nuclease is a multi-turnover Cas nuclease or a high-fidelity variant. Some exemplary CRISPR nucleases are further described in, e.g., Yourik et al. Staphylococcus aureus Cas9 is a multiple-turnover enzyme. RNA 25:35-44 (2019) and Kleinstiver et al. High-fidelity CRISPR-Cas9 variants with undetectable genome-wide off-targets. Nature 529:490-495 (2016), which are hereby incorporated by reference in their entirety.

### 2. Guide RNAs and Specifically Degraded Sequences

In some embodiments, the sequence-specifically degrading comprises contacting DNA with one or more guide RNAs that guide a nuclease to the specific sequence or sequences to be degraded. Appropriate guide RNA sequences are chosen based on the specific sequences to be degraded and on the nuclease to be used. For example, if a CRISPR nuclease is used, one or more appropriate guide RNAs that are recognized by the CRISPR nuclease are used. In some embodiments, single guide RNAs (“sgRNAs”) or other types of fused or truncated CRISPR guide RNAs are used with the appropriate CRISPR nuclease. Exemplary guide RNAs are described in, e.g., Fu et al. Improving CRISPR-Cas nuclease specificity using truncated guide RNAs. Nat. Biotechnol. 32:279-284 (2014), which is hereby incorporated by reference in its entirety.

In some embodiments, the one or more guide RNAs comprise one or more modifications. In some such embodiments, the guide RNAs comprise a modified internucleoside linkage. In some embodiments, the modified internucleoside linkage is a phosphorothioate internucleoside linkage. In some embodiments, the guide RNAs comprise a modified sugar. In some embodiments, the modified sugar comprises a 2′-substitution. In some embodiments, the 2′-substitution is a 2′-fluoro, 2′-O-methoxyethyl, 2′-O-alkyl substitution, or a 2′-hydroxy substitution. In the context of a guide RNA, a sugar comprising a 2′-hydroxy substitution, such as a 2′-deoxyribosyl sugar, is a modified sugar. In some embodiments, the 2′-O-alkyl substitution is a 2′-O-methyl substitution. In some embodiments, the modified sugar is a bicyclic sugar. In some embodiments, the bicyclic sugar is a LNA, cEt, or ENA sugar. In some embodiments, the modified sugar is a linear sugar. In some embodiments, the linear sugar is a UNA sugar.

A guide RNA that is configured to bind to a certain sequence comprises a portion that specifically binds to the certain sequence. In some embodiments, a portion of each guide RNA specifically binds to a target region or a portion of a target region of DNA. In some embodiments, a portion of each guide RNA specifically binds to a sequence of DNA comprising a modification, wherein the modified version of the sequence is prevalent in cfDNA obtained from healthy subjects.

In some embodiments, the sequence-specific degrading comprises contacting DNA with a plurality of guide RNAs. In some embodiments, each unique guide RNA specifically binds to a different member of a plurality of specific sequences, if present, in a sample or subsample thereof. In some embodiments, the plurality of guide RNAs comprises guide RNAs that specifically bind to sequences modified in cfDNA obtained from healthy subjects. In some embodiments, the plurality of guide RNAs comprises guide RNAs that specifically bind to sequences comprising a CpG motif that is methylated in cfDNA obtained from a healthy subject. In some embodiments, the plurality of sequences degraded is 1-100 million sequences. In some embodiments, the plurality of sequences degraded is 20-250,000; 20-100,000; 20-10,000; 20-1,000; or 20-100 sequences. In some embodiments, the plurality of sequences degraded is 50-100,000; 50-10,000; 50-1,000; or 50-100 sequences. In some embodiments, the plurality of sequences degraded is 100-100,000; 100-10,000; or 100-1,000 sequences. In some embodiments, each of the plurality of sequences degraded is a sequence present in a different gene or genetic locus. In some embodiments, sequences that are degraded comprise, or are selected from, repetitive elements (e.g., LINEs such as LINE1; Alu elements; and/or SINEs). In some embodiments, sequences that are degraded comprise sequences known to be modified in cfDNA obtained from healthy subjects. In some embodiments, sequences that are degraded comprise sequences determined to be modified in cfDNA obtained from a healthy subject using existing methods of detecting sites of modifications in DNA sequences.

In some embodiments, methods herein comprise an element or step to deplete unmodified or unmethylated sequences prevalent in cfDNA obtained from healthy subjects. In some such embodiments, the sequence-specific degrading comprises degrading sequences lacking motifs that may be modified or methylated. In some such embodiments, the sequences that are degraded comprise sequences lacking CpG motifs. In some embodiments, the sequence-specific degrading comprises contacting DNA with a plurality of guide RNAs, wherein the plurality of guide RNAs comprises guide RNAs that specifically bind to sequences that are unmethlyated in cfDNA obtained from healthy subjects. In some embodiments, each of the plurality of guide RNAs specifically binds to a sequence prevalent in a form comprising a modification in cfDNA from a healthy subject or to a sequence prevalent in a form lacking the modification in cfDNA from a healthy subject.

B. Degrading DNA with a Methylation-Sensitive Nuclease

In some embodiments, methods herein comprise contacting DNA with a methylation-sensitive nuclease, thereby degrading DNA comprising unmethylated sequences or sequences having low levels of methylation. In some such embodiments, the methylation-sensitive nuclease is a methylation-sensitive restriction enzyme (MSRE), thereby degrading DNA comprising an unmethylated recognition site of the MSRE. Methylation-sensitive nucleases can thus be used in methods herein comprising one or more steps that deplete unmodified or unmethylated sequences that are prevalent in cfDNA from healthy subjects.

In some embodiments, contacting DNA with a methylation-sensitive nuclease is performed before sequence-specifically degrading the DNA with a sequence-specific nuclease. In some embodiments, contacting DNA with a methylation-sensitive nuclease is performed after sequence-specifically degrading the DNA with a sequence-specific nuclease. In some embodiments, contacting DNA with a methylation-sensitive nuclease is performed simultaneously with sequence-specifically degrading the DNA with a sequence-specific nuclease.

C. Partitioning the Sample into a Plurality of Subsamples

Disclosed methods herein comprise analyzing DNA in a sample. In such methods, different forms of DNA (e.g., hypermethylated and hypomethylated DNA) can be physically partitioned based on one or more characteristics of the DNA into a plurality of subsamples. This approach can be used to determine, for example, whether certain sequences are hypermethylated or hypomethylated.

In some embodiments, the partitioning comprises contacting the DNA with an agent that recognizes a modification associated with (e.g., in) the DNA. In some embodiments, the agent that recognizes the modification is an antibody. In some embodiments, the agent is immobilized on a solid support. In some embodiments, the partitioning comprises immunoprecipitation, e.g., using the antibody agent, such as an antibody, immobilized on solid support.

In some embodiments, the modification is methylation, and in some such embodiments, the partitioning comprises partitioning on the basis of methylation level. In some such embodiments, the agent is a methyl binding reagent. In some embodiments, the methyl binding reagent specifically recognizes 5-methylcytosine. In some such embodiments, the agent is a hydroxymethyl binding reagent. In some embodiments, the methyl binding reagent specifically recognizes 5-hydroxymethylcytosine, biotinylated 5-hydroxymethylcytosine, glucosylated 5-hydroxymethylcytosine, or sulfonylated 5-hydroxymethylcytosine. In some embodiments, the partitioning comprises partitioning on the basis of binding to a protein comprising contacting the sample comprising the DNA with a binding reagent specific for the protein. In some such embodiments, binding reagent specifically binds a methylated protein, an acetylated protein, such as a methylated or acetylated histone. In some embodiments, the binding reagent specifically binds an unmethylated or unacetylated protein epitope.

In some embodiments, the modification is hydroxymethylation, and in some such embodiments, the partitioning comprises partitioning on the basis of hydroxymethylation level. In some such embodiments, the agent is a hydroxymethyl binding reagent, such as an antibody. In some embodiments, the hydroxymethyl binding reagent (e.g., antibody) specifically recognizes 5-hydroxymethylcytosine (5-hmC). In some embodiments, a modification such as hydroxymethylation is labeled (e.g., biotinylated, glucosylated, or sulfonated) before being contacted with an agent that recognizes the labeled form of the modification. For example, 5-hmC can be enzymatically glucosylated and then partitioned based on binding to J-binding protein 1. Exemplary methods of labeling and/or partitioning 5-hmC are provided, e.g., in Song et al., Nat. Biotech. 29:68-72 (2010); Ko et al., Nature 468:839-843 (2010); and Robertson et al., Nucleic Acids Res. 39:e55 (2011).

Where immunoprecipitation is used and involves an antibody that recognizes single-stranded DNA, the DNA may be converted to double-stranded form by complementary strand synthesis before the sequence-specific degrading step. Such synthesis may use an adapter as a primer binding site, or can use random priming.

In some embodiments, a sample comprising DNA is partitioned into a plurality of subsamples. In some embodiments, the plurality of partitioned subsample comprises two subsamples, a first subsample and a second subsample. In some embodiments, the plurality of partitioned subsamples comprises three subsamples, a first subsample, second subsample, and third subsample. In some embodiments, the methods comprise a partitioning step that is performed before a sequence-specific degradation step. Some such embodiments comprise contacting the first subsample with a sequence-specific nuclease. Some such embodiments comprise contacting the first subsample with a sequence-specific nuclease and contacting the second subsample with a sequence-specific nuclease. In some embodiments comprising a third partitioned subsample, the third subsample comprises DNA associated with a modification in a greater proportion than it is associated with DNA in the second subsample and in a lesser proportion that it is associated with DNA in the first subsample. In some embodiments, the methods comprise a partitioning step that is performed before sequence-specifically degrading DNA and before contacting the DNA with a methylation-sensitive nuclease.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable target regions are analyzed to determine whether they show differential methylation characteristic of tumor cells or cells of a type that does not normally contribute to the DNA sample being analyzed (such as cfDNA), and/or particular immune cell types.

In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Exemplary partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as Hpall, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents or binding reagents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning is performed after contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by cluting fractions by increasing the NaCl concentration.

In some embodiments, bound DNA is cluted by contacting the antibody or MBD with a protease, such as proteinase K. This may be performed instead of or in addition to elution steps using NaCl as discussed herein.

Examples of agents that recognize a modified nucleobase contemplated herein include, but are not limited to:
(a) MeCP2 and MBD2 are proteins that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOX13 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads. In some embodiments, the partitions of DNA are desalted and concentrated in preparation for enzymatic steps of library preparation.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise capturing at least a first set of target regions from the first pool, e.g., wherein the first pool is as set forth in any of the embodiments herein. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this capture step. In some embodiments, capturing the first set of target regions from the first pool comprises contacting the DNA of the first pool with a first set of target-specific probes. In some embodiments, the first set of target-specific probes comprises target-binding probes specific for the sequence-variable target regions. In some embodiments, the first set of target-specific probes comprises target-binding probes specific for the sequence-variable target regions, hypomethylation variable target regions and/or fragmentation variable target regions.

In some embodiments, the methods comprise capturing a second set of target regions or plurality of sets of target regions from the second pool, e.g., wherein the first pool is as set forth in any of the embodiments herein. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is captured from the second pool but not from the first pool. In some embodiments, a plurality of hypermethylation variable target regions are captured from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

### D. Adapter Ligation or Addition; Tagging

In some embodiments, the disclosed methods comprise adding adapters to DNA. In some embodiments, adapters are added to the DNA before sequence-specifically degrading sequences of the DNA. In some embodiments, adapters are added to the DNA after paritionining and before the sequence-specific degrading. In some embodiments, adapters are added to the DNA before partitioning and before the sequence-specific degrading. In some embodiments, adapters may be added or to DNA concurrently with an amplification procedure, e.g., by providing the adapters in a 5′ portion of a primer (where PCR is used, this can be referred to as library prep-PCR or LP-PCR), before, of after an amplification step. In some embodiments, adapters are added by other approaches. In some such methods, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified. In some embodiments, end repair of the DNA is performed prior to addition of adapters.

In some embodiments, following attachment of adapters, the nucleic acids are subject to amplification. The amplification can use, e.g., universal primers that recognize primer binding sites in the adapters.

In some embodiments, the DNA is linked at both ends to Y-shaped adapters including primer binding sites and tags. In some such embodiments, the DNA is amplified.

In some embodiments, unwanted adapter dimers may form. In some such embodiments, the methods herein comprise sequence-specifically degrading sequences comprising barcode-containng adapter dimer junctions. In some such embodiments, the plurality of guide RNAs comprises a plurality of guide RNAs configured to specifically bind to each possible adapter dimer junction. In such embodiments, the plurality of guide RNAs comprises at least one guide RNA comprising a sequence that is complementary to each one of the possible adapter dimer junction sequences.

Tagging DNA molecules is a procedure in which a tag is attached to or associated with the DNA molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In some such embodiments, the tag comprises one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites. In some embodiments, adapters include the same primer binding site.

In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. In some embodiments comprising barcode attachment, e.g., randomly, to both ends of a molecule, the combination of barcodes, together, constitutes a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by U.S. patent application Ser. Nos. 20010053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, two or more populations, samples, subsamples, or partitions are differentially tagged. Tags can be used to label the individual DNA populations so as to correlate the tag (or tags) with a specific population or partition. In some embodiments, a single tag can be used to label a specific population or partition. In some embodiments, multiple different tags can be used to label a specific population or partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after tagging, the molecules may be pooled for sequencing in a single run. In some embodiments, a sample tag is added to the molecules, e.g., in a step subsequent to addition of other tags and pooling. Sample tags can facilitate pooling material generated from multiple samples for sequencing in a single sequencing run.

In some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules based on one or more characteristics, the final tagged molecules in the library may no longer possess that characteristic. For example, while single stranded DNA molecules may be partitioned and/or tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a pooled DNA level. Tags are used to sort reads from different partitions. Analysis can include in silico analysis to determine genetic and epigenetic variation (one or more of methylation, chromatin structure, etc.) using sequence information, genomic coordinates length, coverage, and/or copy number.

### E. Amplification

In some embodiments, DNA is amplified. For example, DNA flanked by adapters added to the DNA as described herein can be amplified by PCR or other amplification methods. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence based amplification, and self-sustained sequence based replication.

In some embodiments, detecting the presence or absence of one or more DNA sequences comprises amplification, such as embodiments comprising qPCR or digital PCR. Some such embodiments comprising targeted detection of DNA sequences using qPCR or digital PCR do not comprise standard DNA library preparation steps, such as adapter ligation or tagging. In some embodiments, the qPCR can be a library-wide qPCR (i.e., not a targeted PCR). In such embodiments, standard DNA library preparation steps, such as adapter ligation or tagging, are performed prior to contacting the DNA with a methylation-sensitive nuclease (e.g., contacting the DNA with an MSRE) and/or prior to the sequence-specific degrading (e.g., using CRISPR-Cas9 depletion).

In some embodiments, dsDNA ligations with T-tailed and C-tailed adapters can be performed, which result in amplification of at least 50, 60, 70 or 80% of double stranded nucleic acids before linking to adapters.

### F. Detecting; Sequencing

In some embodiments, the detection of the presence of absence of DNA sequences comprises sequencing. In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), enzymatic methyl sequencing (EM-Seq), Tet-assisted pyridine borane sequencing (TAPS), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously

In some embodiments, DNA is sequenced in a modification-sensitive manner (i.e., detecting and/or distinguishing unmodified and modified nucleobases). For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine. Sec, e.g., Schatz., Nature Methods. 14 (4): 347-348 (2017); Weirather J L, et al., F1000Research, 6:100, 2017; and U.S. Pat. No. 9,150,918. Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “ID” reads (“template” and “complement”) by removing the adaptor. The consensus sequence of two “ID” reads is a “2D” read with a higher accuracy.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, the sequencing comprises targeted sequencing in which one or more genomic regions of interest are sequenced. In some such embodiments, the genomic regions of interest comprise regions present one or more genes selected from Tables 1, 2, 3, 4, and/or 5. In some such embodiments, DNA sequences that do not comprise regions of interest are not sequenced. Some embodiments comprise non-targeted sequencing, e.g., all genomic regions of the DNA in a treated sample or subsample are sequenced, or genomic regions are randomly chosen for sequencing. In other embodiments, detecting DNA the presence or absence of sequences comprises sequencing DNA that is not enriched for genomic regions of interest (non-targeted sequencing), e.g., wherein detectable sequences are obtained in a substantially unbiased manner.

In some embodiments, a sequencing step is performed on a library comprising one or more captured target region sets, which may comprise any one or more of the target region sets described herein. In some embodiments, a sequencing step is performed on a library comprising a subsample that has not undergone a capture/enrichment step (e.g., a whole genome subsample). In some embodiments, target regions may be captured from the first subsample and the second subsample and then sequenced. In other embodiments, target regions may be captured from the first subsample and combined with the second subsample after processing, such as after a step of contacting the DNA of the first sample with a methylation-sensitive nuclease and after a step of attaching one or more tags to the DNA of the first and/or second subsamples. In yet other embodiments, target regions may be captured from the second subsample and combined with the first subsample after processing, such as after a step of contacting the DNA of the first sample with a methylation-sensitive nuclease and after a step of attaching one or more tags to the DNA of the first and/or second subsamples. In other embodiments, both the first and second subsamples may be processed (such as tagged with one or more tags) and combined without undergoing capture/enrichment.

The sequencing reactions can be performed on one or more forms of nucleic acids at least one of which is known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

In some embodiments, sequences that were not cleaved during the degrading step are sequenced. In some embodiments, less than 50%, 40%, 30%, 20%, 10%, 5%, 4%, 3%, 2%, or 1% of sequences that were cleaved during the degrading step are sequenced.

In some embodiments, the sequencing comprises generating a plurality of sequencing reads and mapping the plurality of sequencing reads to one or more reference sequences (such as one or more human reference sequences) to generate mapped sequence reads. In some embodiments, at least a portion of the DNA, RNA, or cDNA generated from the RNA of at least a first and second subsample is sequenced in the same sequencing cell.

### G. Target Region Sets

In some embodiments, certain genomic regions of interest are detected and/or enriched. The genomic regions of interest may comprise one or more target region sets. In some embodiments, target region sets comprise variations that are not prevalent in cfDNA from healthy subjects or not prevalent in DNA obtained from healthy tissue regions. In some embodiments, target region sets comprise variations present in healthy cells but not normally present in the sample type, such as a blood sample. In some embodiments, the variations are present in aberrant cells (e.g., hyperplastic, metaplastic, or neoplastic cells). Exemplary target region sets include sequence-variable target region sets, epigenetic target region sets. In some embodiments, the first subsample is enriched for one or more target region sets before sequence-specifically degrading the plurality of DNA sequences in the first subsample that comprise the modification and are prevalent in cell-free DNA. This can be useful where an enrichment procedure results in off-target sequences being present in the enriched DNA, in that the degrading step can then reduce or eliminate the amount of off-target sequences so as to conserve capacity or maximize the efficiency of downstream steps such as sequencing.

In some embodiments, a first target region set is detected, comprising at least epigenetic target regions. In some embodiments, the epigenetic target regions detected in a first subsample comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions show type-specific hypermethylation in healthy cfDNA from one or more related cell or tissue types. Without wishing to be bound by any particular theory, the presence of cancer cells may increase the shedding of DNA into the bloodstream (e.g., from the cancer and/or the surrounding tissue). As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, the methods herein comprise detecting a second captured target region set from a sample or second subsample, comprising at least epigenetic target regions. In some embodiments, the second epigenetic target region set comprises hypomethylation variable target regions. In some embodiments, the hypomethylation variable target regions are CpG-containing regions that are methylated or have high methylation in cfDNA from healthy subjects (e.g., above-average methylation relative to bulk cfDNA). Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

Additionally, target region sets may comprise DNA corresponding to a sequence-variable target region set.

### 1. Epigenetic Target Region Sets

In some embodiments, a target region set is or comprises an epigenetic target region set. Epigenetic target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, an epigenetic target region set has a footprint of at least 100 kb, e.g., at least 200 kb, at least 300 kb, or at least 400 kb. In some embodiments, an epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

a. Hypermethylation and Hypomethylation Variable Target Regions

In some embodiments, an epigenetic target region set comprises a hypermethylation variable target region. In some embodiments, the hypermethylation variable target regions are differentially or exclusively hypermethylated in one or more related cell or tissue types. Such hypermethylation variable target regions may be hypermethylated in other cell or tissue types but not to the extent observed in the one or more related cell or tissue types. In some embodiments, the hypermethylation variable target regions show even higher methylation in cfDNA from a diseased cell of the one or more related cell or tissue types.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 1. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 1
Exemplary Hypermethylation Target
Regions based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, genomic regions targeted for sequencing comprise a plurality of loci listed in Table 1, e.g., at least 10%, 20%, 30%, 40%, 50%, 60%, 70%, 80%, 90%, or 100% of the loci listed in Table 1. In some embodiments, genomic regions are captured using probes. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5 (5): 492-504 (2016); Skvortsova et al., Br. J. Cancer. 94 (10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, genomic regions targeted for sequencing comprise a plurality of loci listed in Table 1 or Table 2, e.g., at least 10%, 20%, 30%, 40%, 50%, 60%, 70%, 80%, 90%, or 100% of the loci listed in Table 1 or Table 2.

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, an epigenetic target region set comprises a hypomethylation variable target region. In some embodiments, the hypomethylation variable target regions are exclusively hypomethylated in one or more related cell or tissue types. Such hypomethylation variable target regions may be hypomethylated in other cell or tissue types but not to the extent observed in the one or more related cell or tissue types.

In some embodiments, where different epigenetic target regions are captured, the epigenetic target regions comprise hypermethylation and/or hypomethylation variable target regions.

Further exemplary hypermethylation variable target regions and hypomethylation variable target regions useful for distinguishing between various cell types have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dec.blueprint-epigenome.cu.

b. CTCF Binding Regions

In some embodiments, an epigenetic target region set comprises CTCF binding regions. CTCF is a DNA-binding protein that contributes to chromatin organization and often colocalizes with cohesin. Perturbation of CTCF binding sites has been reported in a variety of different cancers. See, e.g., Katainen et al., Nature Genetics, doi: 10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Thus, perturbations of CTCF binding result in variation in the fragmentation patterns of cfDNA. As such, CTCF binding sites are a type of fragmentation variable target region.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

c. Transcription Start Sites

In some embodiments, an epigenetic target region set comprises variable transcription start sites. Transcription start sites may show perturbations in neoplastic cells. For example, nucleosome organization at various transcription start sites in healthy cells of the hematopoietic lineage-which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to cfDNA that is typical in healthy subjects. Perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34 (Database issue): D86-D89 (2006), which is incorporated herein by reference. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

d. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1.

e. Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### 2. Sequence-Variable Target Region Sets

In some embodiments, a target region set is or comprises a sequence-variable target region set. Sequence-variable target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The sequence-variable target region set may also comprise one or more control regions, e.g., as described herein. In some embodiments, a sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Examples of listings of genomic locations of interest may be found in, e.g., Table 3 and Table 4 herein. In some embodiments, a sequence-variable target region set comprises portions of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set comprises at least 5, at least 10, at least 15, at least 20, at least 25, at least 30, at least 35, at least 40, at least 45, at least 50, at least 55, at least 60, at least 65, or 70 of the SNVs of Table 3. In some embodiments, a sequence-variable target region set comprises portions of at least 1, at least 2, at least 3, at least 4, at least 5, or 6 of the fusions of Table 3. In some embodiments, a sequence-variable target region set comprises at least portions of at least 1, at least 2, or 3 of the indels of Table 3. In some embodiments, a sequence-variable target region set comprises portions of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, a sequence-variable target region set comprises at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, a sequence-variable target region set comprises portions of at least 1, at least 2, at least 3, at least 4, at least 5, or 6 of the fusions of Table 4. In some embodiments, a sequence-variable target region set comprises at least portions of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. Each of these genomic locations of interest may be identified as a backbone region or hot-spot region for a given panel. Table 5 shows an example listing of hot-spot genomic locations of interest. In some embodiments, a sequence-variable target region set comprises portions of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5. Each hot-spot genomic region is listed with the associated gene, chromosome on which it resides, the start and stop position of the genome representing the gene's locus, the length of the gene's locus in base pairs, the exons covered by the gene, and the critical feature (e.g., type of mutation) of a given genomic region of interest.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | \|311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

Examples of listings of target regions of interest may also be found in WO 2020/160414, e.g., at Table 4. Additional examples include loci disclosed in Gale et al., PLOS One 13: c0194630 (2018), incorporated herein by reference, which describes a panel of 35 cancer-related gene targets: AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1. In some embodiments, the sequence-variable target region set comprises target regions from at least 10, 20, 30, or 35 cancer-related genes, such as the cancer-related genes listed herein and in WO 2020/160414.

In some embodiments, the sequence-variable target region set has a footprint of at least 50 kbp, e.g., at least 100 kbp, at least 200 kbp, at least 300 kbp, or at least 400 kbp. In some embodiments, the sequence-variable target region set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

### H. Subjects

In some embodiments, the DNA (e.g., cfDNA) is obtained from a subject (e.g., a test subject) having a cancer or a precancer. In some embodiments, the subject has a stage I cancer, stage II cancer, stage III cancer, or stage IV cancer. In some embodiments, the DNA from the subject is obtained and/or derived from a sample obtained from the subject. In some embodiments, the DNA is obtained from a subject suspected of having a canceror a precancer. In some embodiments, the DNA is obtained from a subject having a tumor. In some embodiments, the DNA is obtained from a subject suspected of having a tumor. In some embodiments, the DNA is obtained from a subject having neoplasia. In some embodiments, the DNA is obtained from a subject suspected of having neoplasia. In some embodiments, the DNA is obtained from a subject in remission from a tumor, cancer, or neoplasia (e.g., following chemotherapy, surgical resection, radiation, or a combination thereof). In any of the foregoing embodiments, the precancer, cancer, tumor, or neoplasia or suspected precancer, cancer, tumor, or neoplasia may be of the bladder, head or neck, lung, colon, rectum, kidney, breast, prostate, skin, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject. In any of the foregoing embodiments, the subject may be a test subject.

### I. Samples

A sample can be any biological sample isolated from a subject. A sample can be a bodily sample. Samples can include body tissues or fluids, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, gingival crevicular fluid, bone marrow, pleural effusions, pleura fluid, cerebrospinal fluid, saliva, mucous, sputum, semen, sweat, and urine. Samples are preferably body fluids, particularly blood and fractions thereof, cerebrospinal fluid, pleura fluid, saliva, sputum, or urine. A sample can be in the form originally isolated from a subject or can have been subjected to further processing to remove or add components, such as cells, or enrich for one component relative to another.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, post-translation modifications (PTMs) of chromatin, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

Reference or control molecules can be added to or spiked into a sample as a control or normalization standard. For example, a certain amount of modified DNA from a species other than the species of the subject from which the sample was obtained or synthetic nucleic acids comprising certain modifications may be added to the sample. In some embodiments, the reference or control molecules are distinguishable from the molecules originally present in the sample. In some embodiments, the detected DNA sequences are normalized to the reference or control molecules.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

In some embodiments, a plurality of first subsamples (e.g., from different subjects, and/or which have been distinguishably tagged with sample tags) are pooled before sequence-specifically degrading the plurality of DNA sequences that comprise the modification and are prevalent in cell-free DNA. This approach can reduce costs, e.g., in that less reagent may be needed per subsample treated.

### J. Analysis

The present methods can be used to diagnose or classify conditions in a subject. In some embodiments, the condition is cancer or precancer. In some embodiments, the condition is characterized (e.g., staging cancer or determining heterogeneity of a cancer), response to treatment of a condition is monitored, or prognosis risk of developing a condition or subsequent course of a condition is determined. The present disclosure can also be useful in determining the efficacy of a particular treatment option. Successful treatment options may decrease the amount of detected DNA sequences associated with a cancer in a subject's blood as there may be fewer cancer cells to shed DNA. In other examples, this may not occur. In another example, certain treatment options may be correlated with genetic profiles of cancers over time. This correlation may be useful in selecting a therapy

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, colon cancers, prostate cancers, thyroid cancers, bladder cancers, head and neck cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial ancuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In some embodiments, a method described herein comprises detecting the presence or absence of nucleic acids, such as DNA, produced by a tumor (or neoplastic cells, or cancer cells) or by precancer cells.

Information and data generated by the methods disclosed herein can also be used for characterizing a specific form of cancer. Cancers are often heterogeneous in both composition and staging. The methods disclosed herein may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of a condition in a subject. Such methods can include, e.g., generating an aggregate profile of extracellular nucleic acids derived from the subject, wherein the aggregate profile comprises a plurality of data resulting from various nucleic acid analyses. In some embodiments, the aggregate profile comprises epigenetic and mutation analyses. In some embodiments, an aggregate profile comprises a summation of information derived from different cells in a heterogeneous disease. This summation may comprise structural variation identities and levels, copy number variation, epigenetic variation, or other mutation analyses.

The present methods can be used to diagnose, prognose, monitor or observe pre-cancers, cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for analyzing DNA comprises the following steps:

1. Preparing an extracted DNA sample (e.g., extracting blood plasma DNA from a human sample).
2. Partitioning the sample into a plurality of subsamples based on the methylation status of the DNA.
3. Applying different tags and NGS-enabling adapter sequences to each partition.
4. Sequence-specifically degrading sequences of the DNA of at least one subsample using a modification-independent sequence-specific nuclease and a plurality of guide RNAs.
5. Amplifying the DNA using adapter-specific DNA primer sequences.
6. Sequencing the DNA on an NGS instrument.
7. Performing bioinformatics analysis of the NGS data, wherein the tags are used to identify unique molecules and deconvolute the sample into molecules that were differentially partitioned.

In some embodiments, detecting the presence, levels, or absence of DNA sequences facilitates disease diagnosis or identification of appropriate treatments. In some embodiments, the presence of or a change in the levels of one or more sequences is indicative of the presence of a disease or disorder in a subject, such as cancer or precancer, or other disorder that causes changes in nucleic acids relative to a healthy subject.

### III. Additional Features of Some Disclosed Methods

A. Procedures that Affect a First Nucleobase in DNA Differently from a Second Nucleobase in DNA

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA after sequence-specifically degrading the DNA. Alternatively, the DNA is subjected to the procedure before the sequence-specific degradation, wherein the sequence-specific degradation comprises the use of a plurality of guide RNAs comprising guide RNAs configured to specifically bind to DNA comprising converted nucleobases.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosinc (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA comprises bisulfite conversion. Performing bisulfite conversion can facilitate identifying positions containing mC or hmC using the sequence reads. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosinc, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, thus facilitates identifying positions containing mC or hmC using the sequence reads obtained from the exemplary sample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149:1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

In some embodiments, protection of hmC (e.g., using βGT) can be combined with Tet-assisted conversion with a substituted borane reducing agent. Performing TAPSβ conversion can facilitate distinguishing positions containing unmodified C or hmC on the one hand from positions containing mC using the sequence reads. hmC can be protected as noted above through glucosylation using βGT, forming ghmC. Treatment with a TET protein such as mTet1 then converts mC to caC but does not convert C or ghmC. caC is then converted to DHU by treatment with pic-borane or another substituted borane reducing agent such as borane pyridine, tert-butylamine borane, or ammonia borane, also without affecting unmodified C or ghmC. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO₄) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-BGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample converts a modified nucleoside. In some embodiments, the conversion procedure which converts a modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA. Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

In some embodiments, the procedure that affects a first nucleobase in the DNA differently from a second nucleobase in the DNA comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form β-6-azide-glucosyl-5-hydroxymethylcytosinc and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63:711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N⁶-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

### B. Capturing DNA; Capture Moieties

In some embodiments, methods herein comprise capturing or enriching nucleic acid molecules comprising sequences present in a target region set for subsequent analysis. Such enrichment or capture may be performed on any sample or subsample described herein using any suitable approach known in the art. In some embodiments, the capturing comprises contacting the DNA with probes specific for such target regions. In some embodiments, the probes comprise an oligonucleotide and a capture moiety, such as biotin or the other examples noted below. The probes can have sequences selected to tile across a panel of regions, such as genes.

Methods comprising DNA capture using probes comprising a capture moiety, such as target-specific probes labeled with biotin, may also comprise a second moiety or binding partner that binds to the capture moiety, such as streptavidin. In some embodiments, a capture moiety and binding partner can have higher and lower capture yields for different sets of probes, such as those used to capture a sequence-variable target region set and an epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. In some embodiments, a capture moiety that is attached to an analyte is captured by its binding partner which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, target regions are captured from an aliquot, portion, or subsample of a sample (e.g., a sample that has undergone attachment of adapters and amplification), while a step of partitioning the DNA may be performed on a separate aliquot, portion, or subsample of the sample. Enriching for or capturing DNA comprising target regions may comprise contacting the DNA with a first or second set of target-specific probes. Such target-specific probes may have any of the features described herein for sets of target-specific probes, including but not limited to in the embodiments set forth herein and the sections relating to probes herein. Capturing may be performed on one or more subsamples prepared during methods disclosed herein. In some embodiments, DNA is captured from a first subsample or a second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step or steps may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization.

In some embodiments, methods described herein comprise capturing a plurality of target region sets of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation.

In some embodiments, the captured DNA is amplified. In various embodiments, the methods further comprise sequencing the captured DNA, e.g., to different degrees of sequencing depth for the epigenetic and sequence-variable target region sets, consistent with the discussion herein. In some embodiments, RNA probes are used. In some embodiments, DNA probes are used. In some embodiments, single stranded probes are used. In some embodiments, double stranded probes are used. In some embodiments, single stranded RNA probes are used. In some embodiments, double stranded DNA probes are used.

In some embodiments, a capturing step is performed with probes for a sequence-variable target region set and probes for an epigenetic target region set in the same vessel at the same time, e.g., the probes for the sequence-variable and epigenetic target region sets and capture probes are in the same composition. This approach provides a relatively streamlined workflow.

In some embodiments, a collection of target-specific probes is used in methods comprising capturing DNA described herein. In some embodiments, the collection of target-specific probes comprises target-binding probes specific for one or more target region sets. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

### C. Computer Systems

Methods of the present disclosure can be implemented using, or with the aid of, computer systems. For example, such methods may comprise: subjecting DNA in a sample or subsample to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity; sequence-specifically degrading target DNA sequences; and sequencing remaining DNA in a manner that distinguishes the first nucleobase from the second nucleobase in the DNA.

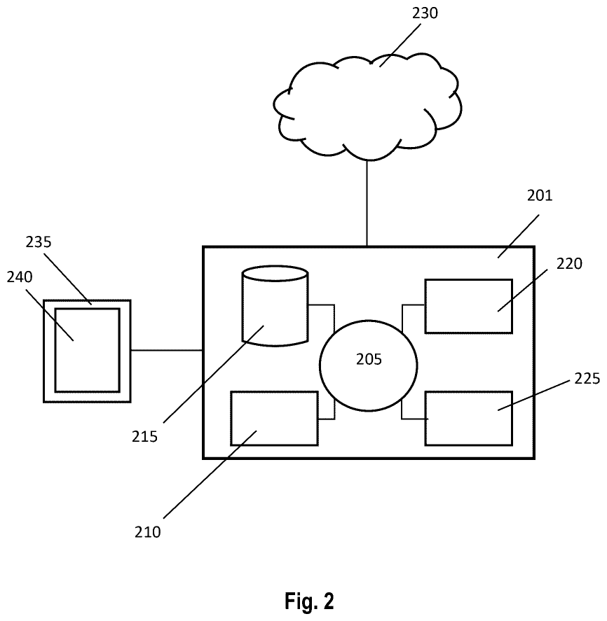

FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method comprising: collecting cfDNA from a test subject; partitioning the DNA into a plurality of subsamples based on the level of a modification associated with (e.g., in) the DNA and/or contacting the sample with a methylation-sensitive nuclease; sequence-specifically degrading sequences of the DNA using a modification-dependent sequence-specific nuclease; sequencing the remaining cfDNA; obtaining a plurality of sequence reads generated by a nucleic acid sequencer from sequencing the cfDNA molecules; mapping the plurality of sequence reads to one or more reference sequences to generate mapped sequence reads; and processing the mapped sequence reads to determine the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurosc, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### D. Applications

### 1. Cancer and Other Diseases

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of copy number variation, rare mutations, and/or cancer-related epigenetic signatures (such as hypermethylated regions or hypomethylated regions) detected in a subject's blood (such as in cfDNA isolated from a blood sample (e.g., a whole blood sample, a leukapheresis sample, a buffy coat sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject.

In some embodiments, the present methods are used for screening for a cancer, or in a method for screening cancer. For example, the sample can be a sample from a subject who has not been previously diagnosed with cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at www.dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial ancuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Methods herein can also be used for characterizing a specific form of cancer. Cancers are often heterogeneous in both composition and staging. Characterization of specific sub-types of cancer may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogencity of an abnormal condition in a subject. Such methods can include, e.g., generating a profile of cfDNA derived from the subject. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogencity may comprise multiple foci of disease. Again, in the example of cancer, there may be multiple tumor foci, perhaps where one or more foci are the result of metastases that have spread from a primary site. The tissue(s) of origin can be useful for identifying organs affected by the cancer, including the primary cancer and/or metastatic tumors.

In some embodiments, a method described herein comprises detecting a presence or absence of a nucleic acid originating or derived from a tumor cell at a preselected timepoint following a previous cancer treatment of a subject previously diagnosed with cancer. The method may further comprise determining a cancer recurrence score that is indicative of the presence or levels of DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, vclocardio facial syndrome, WAGR syndrome, Wilson disease, or the like.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. In some embodiments, quantities of each of a plurality of cell types, such as immune cell types, are determined based on sequencing and analysis (such as determination of epigenetic and/or genomic signatures) of DNA (such as cfDNA) isolated from at least one sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample.

Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells. As discussed herein, comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed herein may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Test Subject as being a Candidate for a Subsequent Cancer Treatment

In some embodiments, a method provided herein is a method of determining a risk of cancer recurrence in a subject. In some embodiments, a method provided herein is a method of classifying a subject as being a candidate for a subsequent cancer treatment.

Any of such methods may comprise collecting nucleic acids (e.g., DNA or RNA originating or derived from a tumor cell) from the subject diagnosed with the cancer at one or more preselected timepoints following one or more previous cancer treatments to the subject. The subject may be any of the subjects described herein. The DNA may comprise cfDNA. The DNA may be DNA, such as cfDNA, from a blood sample (e.g., a whole blood sample). The DNA may comprise DNA obtained from a tissue sample.

Any of such methods may comprise contacting the sample or a subsample thereof with a modification-independent sequence-specific nuclease according to any of the embodiments as described herein. Any of such methods may comprise sequencing DNA molecules, whereby a set of sequence information is produced. Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

In any of such methods, the previous cancer treatment may comprise surgery, administration of a therapeutic composition, and/or chemotherapy.

Methods of determining a risk of cancer recurrence in a subject may comprise determining a cancer recurrence score that is indicative of the presence or absence, or amount, of genomic regions of interest and target regions originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises target region set sequences, and determining the cancer recurrence score may comprise determining at least a first subscore indicative of the levels of particular cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, from a blood sample (e.g., a whole blood sample), and/or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a Cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment. In some embodiments, the cancer is any one of the types of cancer described elsewhere herein.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample, e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to subjects. In some embodiments, determination of the levels of particular nucleic acids facilitates selection of appropriate treatment. In some embodiments, the subject has a given disease, disorder, or condition, e.g., any of the conditions described elsewhere herein.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### IV. Kits

Also provided are kits comprising compositions as described herein. The kits can be useful in performing the methods described herein. In some embodiments, a kit comprises first reagents for sequence-specifically degrading DNA sequences. In some such embodiments, the reagents for sequence-specific degradation comprise a modification-independent sequence-specific nuclease and a plurality of guide RNAs. In some embodiments, the nuclease is a CRISPR nuclease, and the one or more guide RNAs are sgRNAs and/or comprise one or more modifications. In some embodiments, the kit further comprises second reagents for partitioning each sample into a plurality of subsamples as described herein, such as any of the partitioning reagents described elsewhere herein. In some embodiments, the reagents for partitioning comprise an agent that recognizes a modification, such as a modified nucleobase, such as a methylated nucleobase, in DNA. In some embodiments, the agent that recognizes a modified nucleobase in DNA is an antibody. In some embodiments, the modification is a modified cytosine, such as a methyl cytosine or hydroxymethyl cytosine (e.g., a labeled hydroxymethyl cytosine). In some embodiments, the agent that recognizes a modification in DNA is an antibody specific for methyl cytosine in DNA. In some embodiments, the partitioning reagents comprise a solid support. In some embodiments, the kit comprises a methylation-sensitive nuclease. In some embodiments, the kit comprise the first reagents, the second reagents, and, optionally, a methylation-sensitive nuclease and/or other additional elements as discussed elsewhere herein. In some embodiments, a kit comprises instructions for performing a method described herein.

The kit can comprise at least 4, 5, 6, 7, or 8 different adapters having distinct molecular barcodes and/or identical sample barcodes. The adapters may not be sequencing adapters. For example, the adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different adapters having distinct molecular barcodes and identical sample barcodes. The adapters may not be sequencing adapters. The kit can further include a sequencing adapter having a first sequence that selectively hybridizes to the adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached {e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The adapters can be blunt ended and Y-shaped and can be less than or equal to 40 nucleic acid bases in length. Other variations can be found throughout and are applicable to the kit.

Kits may further comprise a plurality of oligonucleotide probes that selectively hybridize to at least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABLl, AKTl, ATM, CDHl, CSFIR, CTNNBl, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Analysis of cfDNA to Detect the Presence or Absence of Tumor in a Subject

The workflow described in this example is illustrated in FIG. 3. A set of patient samples are analyzed by a blood-based NGS assay at Guardant Health (Redwood City, CA, USA) to detect the presence or absence of cancer. cfDNA is extracted from the plasma of these patients. The cfDNA of the patient samples is then combined with magnetic beads conjugated with an MBD protein and appropriate buffers and incubated overnight. Methylated cfDNA is bound to the MBD protein during this incubation. Non-methylated or less methylated DNA is washed away from the beads with buffers containing increasing concentrations of salt. Finally, a high salt buffer is used to wash the heavily methylated DNA away from the MBD protein. The unbound DNA and these washes result in at least three partitions (hypomethylated, residual methylation and hypermethylated partitions) of increasingly methylated cfDNA. The cfDNA molecules in the partitions are cleaned, to remove salt, and concentrated in preparation for the enzymatic steps of library preparation.

After concentrating the cfDNA in the partitions, first adapters are added to the cfDNA by ligation to the 3′ ends thereof. The adapter is used as a priming site for second-strand synthesis using a universal primer and a DNA polymerase. A second adapter is then ligated to the 3′ end of the second strand of the now double-stranded molecules. These adapters contain non-unique molecular barcodes and each partition is ligated with adapters having non-unique molecular barcodes that is distinguishable from the barcodes in the adapters used in the other partitions.

After ligation, the DNA is washed and concentrated prior to enrichment. Once concentrated, the DNA of the hypermethylated partition is combined with a methylation-sensitive restriction enzyme (MSRE) for degradation of nonspecifically partitioned DNA (e.g., unmethylated DNA in the hypermethylated partition). The digested DNA is then combined with a Cas9 nuclease and a plurality of sgRNAs specific for sequences that comprise methyl modifications that are prevalent in DNA from healthy subjects, thus degrading sequences of the hypermethylated partition expected to be present in samples obtained from healthy subjects. The remaining hypermethylated DNA, which is now enriched for sequences not prevalent in methylated form in healthy subjects, is amplified by PCR.

Optionally, the DNA of the hypomethylated partition is amplified by PCR and then washed and concentrated. Once concentrated, the amplified DNA is enriched for target regions of interest by combining it with a salt buffer and biotinylated RNA probes of target region sets comprising probes for sequence-variable target region set probes and/or epigenetic target region set probes. Probes for the epigenetic target region set comprise oligonucleotides targeting hypomethylation variable target regions (e.g., sequences that are hypomethylated in cancer and/or that are not normally hypomethylated in healthy cfDNA), CTCF binding target regions, transcription start site target regions, focal amplification target regions and/or methylation control regions. The mixture is incubated overnight. The biotinylated RNA probes and any hybridized DNA are captured by streptavidin magnetic beads and separated from the uncaptured DNA by a series of salt based washes, thereby enriching the sample. The enriched, hypomethylated DNA may then be combined with the DNA amplified from the hypermethylated DNA enriched for sequences not prevalent in methylated form in healthy subjects.

Sequencing is performed using a next-gen sequencer (e.g., an Illumina NovaSeq sequencer). The sequence reads generated by the sequencer are then analyzed using bioinformatic tools/algorithms. The molecular barcodes are used to identify unique molecules as well as for deconvolution of samples that were differentially partitioned. Sequences of the unique molecules are analyzed to identify genomic alterations such as SNVs, insertions, deletions and fusions that can be called with enough support that differentiates real tumor variants from technical errors (for e.g., PCR errors, sequencing errors). Sequences of molecules from the hypermethylated partition are analyzed independently to detect methylated cfDNA molecules in regions that have been shown to be differentially methylated in cancer compared to normal cells and/or regions that have been shown to be differentially methylated in cell types that do not substantially contribute to cfDNA in healthy individuals. Finally, the results of both analyses are combined to produce a final tumor present/absent call.

### Example 2: Analysis of cfDNA without a Partitioning Step

Cell-free DNA is extracted from a set of patient samples, and adapters are added to the cfDNA, essentially as described in Example 1. After ligation, the DNA is washed and concentrated prior to enrichment. Once concentrated, the DNA is combined with a MSRE to degrade unmethylated DNA. Following MSRE treatment, DNA is then combined with a Cas9 nuclease and a plurality of sgRNAs. Some of the plurality of sgRNAs are specific for sequences that comprise methyl modifications that are prevalent in DNA from healthy subjects. Others of the plurality of sgRNA are specific for sequences that do not comprise CpG dinucleotides. Thus, sequences expected to be highly methlyated in samples obtained from healthy subjects and sequences that do not comprise certain motifs that can be methylated are degraded. The remaining DNA, which is now enriched for sequences not prevalent in methylated form in healthy subjects, is amplified by PCR.

After this enrichment of sequences not prevalent in methylated form in healthy subjects, an aliquot of the enriched sample is sequenced using a next-gen sequencer (e.g., an Illumina NovaSeq sequencer). The sequence reads generated by the sequencer are then analyzed using bioinformatic tools/algorithms. The molecular barcodes are used to identify unique molecules. A tumor present/absent call is made based at least in part on the presence and/or amount of methylated cfDNA molecules in regions that have been shown to be differentially methylated in cancer compared to normal cells and/or regions that have been shown to be differentially methylated in cell types that do not substantially contribute to cfDNA in healthy individuals.

### Example 3: Analysis of Single-Site Methylation in cfDNA

A set of patient samples are analyzed by a blood-based NGS assay at Guardant Health (Redwood City, CA, USA) to detect the presence or absence of cancer, as described in Example 1 or Example 2, with the additional step of subjecting the DNA of the sample or partitioned subsample to a procedure that modified a first nucleobase differently than a second nucleobase. For example, the DNA is treated with bisulfite in order to convert unmodified cytosines to thymines prior to the Cas9 treatment. The plurality of sgRNAs comprises sequences specific for sequences comprising the converted thymines in order to degrade sequences that were originally unmethylated. In another example, the DNA is treated with bisulfite after the Cas9 treatment in order to identify the locations of aberrant cytosine methlyations, or of cytosines that are not methylated in sequences prevalent in cfDNA from healthy subjects.

### Example 4: Additional Methods of Analyzing cfDNA

A set of patient samples are analyzed by a blood-based NGS assay at Guardant Health (Redwood City, CA, USA) to detect the presence or absence of cancer, as described in Example 1, 2, or 3, with the additional feature that the plurality of sgRNAs comprises sgRNAs specific for sequences comprising the unique junction created by adapter dimer ligation events and a portion of the molecular barcode.

1. A method of analyzing DNA in a sample, the method comprising:
a) partitioning the sample into a plurality of subsamples by contacting the DNA with an agent that recognizes a modification associated with the DNA, the plurality comprising a first subsample and a second subsample, wherein the first subsample comprises DNA associated with the modification in a greater proportion than the second subsample;
b) sequence-specifically degrading a plurality of DNA sequences in the first subsample that comprise the modification and are prevalent in cell-free DNA (cfDNA) from a healthy subject, comprising contacting the first subsample with a modification-independent sequence-specific nuclease, thereby producing a treated sample; and
c) detecting the presence or absence of one or more DNA sequences associated with the modification in the treated sample.

2. The method of claim 1, wherein the partitioning occurs prior to the degrading.

3. A method of analyzing cfDNA in a sample, the method comprising:
a) contacting the sample or a subsample thereof with a MSRE, thereby degrading DNA comprising an unmethylated recognition site of the MSRE;
b) sequence-specifically degrading a plurality of DNA sequences having methylated sequences that are prevalent in cfDNA from a healthy subject and a plurality of sequences lacking CpG motifs, comprising contacting the sample with a modification-independent sequence-specific nuclease, thereby producing a treated sample; and
c) detecting the presence of absence of one or more DNA sequences in the treated sample.

4. (canceled)

5. (canceled)

6. The method of claim 1, wherein the partitioning comprises partitioning on the basis of methylation level of the DNA.

7. The method of claim 1, wherein the modification is methylation.

8. The method of the immediately preceding claim 7, wherein the methylation comprises cytosine methylation.

9. The method of claim 1, wherein the partitioning comprises partitioning on the basis of hydroxymethylation level of the DNA.

10. The method of claim 9, wherein hydroxymethyls are labeled prior to partitioning, optionally wherein the label comprises a biotin, glucosyl, or sulfonyl.

11. (canceled)

12. (canceled)

13. The method of claim 1, wherein the agent that recognizes a modification associated with the DNA is a methyl, hydroxymethyl, or labeled hydroxymethyl binding reagent is an antibody.

14. The method of claim 13, wherein the methyl binding reagent specifically recognizes 5-methylcytosine, 5-hydroxymethylcytosine, biotinylated 5-hydroxymethylcytosine, glucosylated 5-hydroxymethylcytosine, or sulfonylated 5-hydroxymethylcytosine.

15. (canceled)

16. (canceled)

17. (canceled)

18. (canceled)

19. The method of claim 1, wherein the degrading comprises contacting the second subsample with a modification-independent sequence-specific nuclease, thereby producing a second treated sample.

20. The method of claim 1, comprising contacting one or more of the plurality of subsamples with a methylation-sensitive restriction enzyme (MSRE), thereby degrading DNA comprising an unmethylated recognition site of the MSRE.

21-26. (canceled)

27. The method of claim 1, wherein the modification-independent sequence-specific nuclease is a CRISPR nuclease.

28-33. (canceled)

34. The method of claim 1, wherein the sequence-specifically degrading comprises contacting the DNA with a plurality of guide RNAs.

35-39. (canceled)

40. The method of claim 34, wherein at least one guide RNA specifically binds to a DNA sequence lacking a CpG dinucleotide.

41. The method of claim 34, wherein each guide RNA of the plurality of guide RNAs is configured to specifically bind to a DNA sequence that comprises the modification and is prevalent in cell-free DNA (cfDNA) from a healthy subject.

42. The method of claim 34, wherein each guide RNA of the plurality of guide RNAs is configured to specifically bind to a DNA sequence that comprises the modification and is prevalent in cell-free DNA (cfDNA) from a healthy subject or to a DNA sequence lacking a CpG dinucleotide.

43-45. (canceled)

46. The method of claim 1, wherein the detecting step comprises sequencing.

47-49. (canceled)

50. The method of claim 46, wherein the detecting step comprises sequencing a plurality of target regions in at least one target region set and the at least one target region set comprises target regions that are not prevalent in methylated form in cfDNA from a healthy subject or not prevalent in methylated form in healthy tissue.

51. The method of claim 46, wherein the detecting step comprises sequencing a plurality of target regions in at least one target region set and the at least one target region set comprises target regions that are prevalent in methylated form in tissue that does not substantially contribute to cfDNA in a healthy subject or target regions that are prevalent in methylated form in a cancerous tissue.

52-96. (canceled)
